(19) United States
(12) Patent Application Publication
(10) Pub. No.: US 2025/0206685 A1
(43) Pub. Date: Jun. 26, 2025

(54) PROCESS FOR CONVERTING NAPHTHA WITH PROPANE RECYCLE

(71) Applicant: UOP LLC, Des Plaines, IL (US)

(72) Inventors: Gregory A. Funk, Carol Stream, IL (US); Deng-Yang Jan, Elk Grove Village, IL (US); John J. Senetar, Naperville, IL (US); Scott Lyle Nauert, Chicago, IL (US); Bhogeswararao Seemala, Arlington Heights, IL (US)

(21) Appl. No.: 18/984,810

(22) Filed: Dec. 17, 2024

Related U.S. Application Data
(60) Provisional application No. 63/614,434, filed on Dec. 22, 2023.

(51) Int. Cl. C07C 5/327; C07C 4/04; C07C 4/06; C07C 7/00

(52) U.S. Cl. CPC C07C 5/327; C07C 4/04; C07C 4/06; C07C 7/00

(57) ABSTRACT

A process for converting naphtha is disclosed. The process comprises contacting a naphtha stream and a hydrogen stream with a catalyst in a reactor to produce a light paraffinic stream. The light paraffinic stream is separated into an ethane stream and a propane stream. A propane recycle stream taken from the propane stream is recycled to the reactor.

### FIELD

The field is the conversion of naphtha to paraffins. The field may particularly relate to converting naphtha to ethane.

### BACKGROUND

Light olefin production is vital to the production of sufficient plastics to meet worldwide demand. Dehydrogenation is a process in which light paraffins such as ethane and propane can be dehydrogenated to make ethylene and propylene respectively, typically in the presence of a catalyst. Dehydrogenation can be achieved in either the presence of an oxidant such as oxygen or in the absence of an oxidant. Non-oxidative dehydrogenation is an endothermic reaction which requires external heat to drive the reaction to completion. Propane dehydrogenation (PDH) is a widely practiced example of non-oxidative dehydrogenation to produce propylene from propane. Ethane oxidative dehydrogenation is a newer oxidative process for converting ethane to ethylene which can be conducted at lower temperatures with lower carbon oxide emissions than non-oxidative and thermal cracking processes.

Fluid catalytic cracking (FCC) is another endothermic process that can be tuned to produce substantial propylene. However, not every FCC unit is tuned to make substantial propylene. Also, high propylene FCC units do not recover much ethylene; less than 1% of global ethylene supply comes from FCC.

The great bulk of the ethylene consumed in the production of plastics and petrochemicals such as polyethylene is produced by the thermal cracking of hydrocarbons. Steam is usually mixed with the feed stream to the cracking furnace to reduce the hydrocarbon partial pressure and enhance olefin yield and to reduce the formation and deposition of carbonaceous material in the cracking reactors. The process is therefore often referred to as steam cracking or pyrolysis.

Paraffins with a range of carbon numbers can be thermally cracked to produce olefins including ethane, propane, butanes, and naphtha. Ethane and naphtha feeds are typical due to higher light olefin yield than propane and butane feeds. Ethane feed is used in regions where light hydrocarbon gases are prevalent. In regions, where gas is not abundant, naphtha feed is employed for steam cracking. Naphtha steam cracking has long set the price in the ethylene industry due to higher production cost versus ethane steam cracking. The world does not currently produce enough ethane to supply the growing demand for ethylene. Therefore, regions lacking ethane supply such as Asia and Europe rely mainly on naphtha steam cracking to supply ethylene. Naphtha steam cracking yields only about 30%-35% ethylene with the balance including both relatively high-value by-products comprising propylene, butadiene, and butene-1 and relatively low value by-products comprising pyoil, pygas, and fuel gas. Additional pressures on naphtha steam cracking including minimum production requirements and environmental concerns have led to the withholding of government approvals in certain regions such as China. The ethylene industry needs a more efficient, economical and environmentally friendly route to light olefins from naphtha feeds.

### BRIEF SUMMARY

A process for converting naphtha to paraffins is disclosed. The process comprises contacting a naphtha stream and a hydrogen stream with a catalyst to produce a light paraffinic stream. The light paraffinic stream may be separated into an ethane stream and a propane stream. A propane recycle stream is taken from the propane stream and recycled to the reactor to increase ethane production.

### BRIEF DESCRIPTION OF THE DRAWINGS

FIG. 1 is a schematic drawing of a process for converting naphtha in accordance with an exemplary embodiment of the present disclosure.

FIG. 2 is a schematic drawing of a process for converting naphtha in accordance with another exemplary embodiment of the present disclosure.

### DEFINITIONS

The term “communication” means that fluid flow is operatively permitted between enumerated components, which may be characterized as “fluid communication”.

The term “downstream communication” means that at least a portion of fluid flowing to the subject in downstream communication may operatively flow from the object with which it fluidly communicates.

The term “upstream communication” means that at least a portion of the fluid flowing from the subject in upstream communication may operatively flow to the object with which it fluidly communicates.

The term “direct communication” means that fluid flow from the upstream component enters the downstream component without passing through any other intervening vessel.

The term “indirect communication” means that fluid flow from the upstream component enters the downstream component after passing through an intervening vessel.

The term “bypass” means that the object is out of downstream communication with a bypassing subject at least to the extent of bypassing.

As used herein, the term “predominant” or “predominate” or “predominance” means greater than 50%, suitably greater than 75% and preferably greater than 90%.

The term “Cx” is to be understood to refer to molecules having the number of carbon atoms represented by the subscript “x”. Similarly, the term “Cx-” refers to molecules that contain less than or equal to x and preferably x and less carbon atoms. The term “Cx+” refers to molecules with more than or equal to x and preferably x and more carbon atoms.

The term “column” means a distillation column or columns for separating one or more components of different volatilities. Unless otherwise indicated, each column includes a condenser on an overhead of the column to condense and reflux a portion of an overhead stream back to the top of the column and a reboiler at a bottom of the column to vaporize and send a portion of a bottoms stream back to the bottom of the column. Feeds to the columns may be preheated. The top pressure is the pressure of the overhead vapor at the vapor outlet of the column. The bottom temperature is the liquid bottom outlet temperature. Overhead lines and bottoms lines refer to the net lines from the column downstream of any reflux or reboil to the column. Stripper columns may omit a reboiler at a bottom of the column and instead provide heating requirements and separation impetus from a fluidized inert media such as steam. Stripping columns typically feed a top tray and take main product from the bottom.

The terms “T10” and “T90” are used here to characterize the volatility of a petroleum fraction such as naphtha. T10 and T90 refer to the temperatures for recovery of 10% and 90%, respectively, in distillation of petroleum products at atmospheric pressure using standard method ASTM D86.

As used herein, the term “separator” means a vessel which has an inlet and at least an overhead vapor outlet and a bottoms liquid outlet and may also have an aqueous stream outlet from a boot. A flash drum is a type of separator which may be in downstream communication with a separator that may be operated at higher pressure.

### DETAILED DESCRIPTION

In the proposed process, C3-C8+ hydrocarbon feed stock is first charged to a “Naphtha to Ethane and Propane” (NEP) unit to convert naphtha in the presence of hydrogen into desirable ethane and propane. The ethane produced is fed to an ethylene producing unit. The ethylene producing units provide over 75% yield of ethane to ethylene. The produced propane is fed to a propylene producing unit which provides over 85% yield of propane to propylene. The methane by-product from the naphtha conversion unit and the ethane and propane producing units can be used as a fuel including fuel needed to operate ethylene and propylene producing units which operate at elevated temperatures. Unconverted or under-converted C4+ components in the reactor outlet may be recycled to the reactor inlet for further processing to ethane and propane.

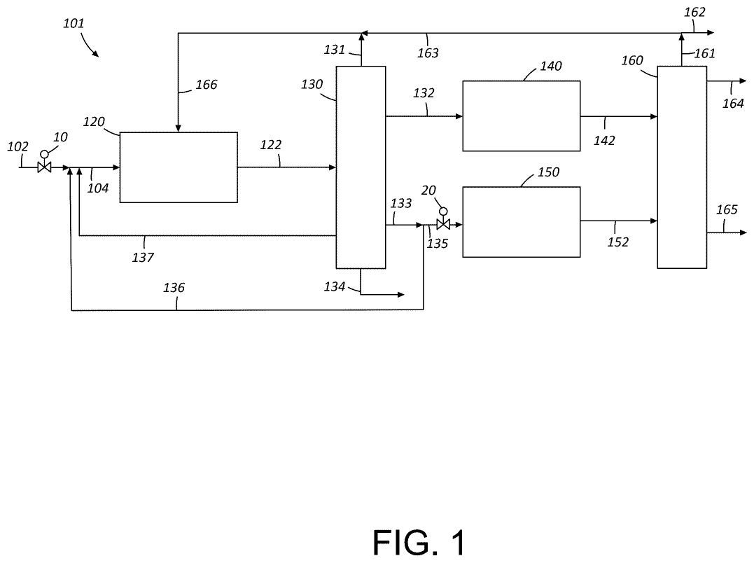

Turning to FIG. 1, an embodiment of the process for converting naphtha 101 is disclosed. In an embodiment, the process 101 comprises a NEP reactor 120, an NEP separation unit 130, an ethane conversion unit 140, a propane conversion unit 150, and a product recovery unit 160. A naphtha stream in line 102 may be combined with a hydrogen stream in line 166 to provide a charge stream in line 104. The charge stream in line 104 may be charged to the NEP reactor 120 to be contacted with an NEP catalyst. As described hereinafter in detail, one or more recycle streams, a paraffins stream in line 137 and a hydrogen stream in line 166 are also recycled to the NEP reactor 120. The naphtha stream may comprise C3 to C8+ hydrocarbons preferably having a T10 between about −10° C. and about 60° C. and a T90 between about 70 and about 180° C. The naphtha feed stream may comprise normal paraffins, iso-paraffins, olefins, naphthenes, and aromatics. In an aspect, the naphtha stream in line 102 may be heated before charging to the NEP reactor 120. In an embodiment, the naphtha stream may be heated to a reaction temperature of about 300° C. to about 600° C., suitably between about 325° C. and about 550° C., and preferably between about 350° C. and about 525° C. Overall, weight space velocity defined herein as hourly mass flow rate of feed in stream 102 divided by total catalyst in all reactors should be between about 0.3 to about 20 hr⁻¹, suitably between about 0.5 and about 10 hr⁻¹ and preferably between about 1 to about 4 hr⁻¹. A total pressure should be about 0.1 to about 3 MPa (abs), preferably greater than 1 MPa (abs). Under these conditions, C2-C4 yield is consistently in an excess of 80 wt %, while methane yield is less than about 16 wt %, suitably below about 14 wt % and typically below about 12 wt % and preferably no more than 10 wt %.

The hydrogen-to-hydrocarbon molar ratio is important to producing ethane and propane. The hydrogen-to-hydrocarbon ratio should be about 0.3 to about 15 and preferably about 0.5 to about 5. In a further embodiment, the hydrogen-to-hydrocarbon molar ratio may typically be no more than 5, suitably be no more than 3 and preferably be no more than 2. Low hydrogen-to-hydrocarbon ratio promotes desired reaction kinetics which are initiated with dehydrogenation. Hydrogen-to-hydrocarbon ratio may range from about 50% to about 500%, suitably no more than 300% and preferably no more than 200%, of stoichiometric requirements to convert naphtha molecules to ethane and/or propane. The molar ratio of hydrogen to hydrocarbon depends on the feed type including paraffin, olefin, naphthene or aromatics, the feed molecular carbon number, and the desired product between predominantly ethane, predominantly propane or ethane and propane of comparable abundance. For example, converting 1 mole of propane to ethane at stoichiometry, the process would require co-feeding 0.5 moles of hydrogen. In practice, the process can operate above or below this stoichiometry of 0.5 such as 0.33 to achieve greater than 40% ethane and less than 15% methane, depending on the process design parameters such as, feed contaminants, reactor type (fixed bed, moving bed, fluidized bed), and regeneration frequency. As the carbon number of feed molecules increases from light naphtha (C4-C7) to full range naphtha (C6-C10) the amount of hydrogen required for the reaction increases. For example, it would require 3.5 moles hydrogen and 2.0 moles of hydrogen to fully convert 1 mole of nonane to ethane and propane, respectively. The disclosed process can operate at three to five times the hydrogen-to-hydrocarbon ratio required to stoichiometrically convert the feed molecules to ethane and propane, respectively. It can also operate at 50% of hydrogen-to-hydrocarbon ratio required to stoichiometrically convert the feed molecules to ethane and propane, respectively. The hydrogen-to-hydrocarbon ratio would also depend on the need to produce petrochemical aromatics such as benzene, toluene and xylene.

The NEP catalyst for converting naphtha to ethane and propane may contain a molecular sieve comprising large or medium pore mouths, that is, comprising 10 or 12 member rings, respectively. Examples of suitable molecular sieves include MFI, MEL, MFI/MEL intergrowth, MTW, TUN, UZM-39, IMF, UZM-44, UZM-54, MWW, UZM-37, UZM-8, UZM-8HS. Examples of suitable molecular sieves further include FER, AHT, AEL (SAPO-11), AFO (SAPO-41), MRE, MFS, EUO-1, TON (ZSM-22), MTT (ZSM-23) and UZM-53. Additional molecular sieves with larger pores include FAU, EMT, FAU/EMT intergrowth, UZM-14, MOR, BEA, UZM-50, MTW, ZSM-12. Additional examples include MSE and UZM-35.

MFI is a suitable NEP catalyst. It will be appreciated that ZSM-5 is an MFI-type aluminosilicate zeolite belonging to the pentasil family of zeolites and having a chemical formula of NaₙAlₙSi₉₆-nO₁₉₂.16H₂O (0<n<10). In various embodiments, the ZSM-5 zeolite may comprise a silica-to-alumina molar ratio of 20 to 1000, 20 to 800, 20 to 600, 20 to 400, 20 to 200 or 20 to 80. In various embodiments, the ZSM-5 zeolite may comprise a crystal size in the range of 10 to 600 nm, 20 to 500 nm, 30 to 450, 40 to 400 nm, or 50 to 300 nm.

The NEP catalyst may comprise a bound zeolite. The binder may comprise an oxide of aluminum, silicon, zinc, titanium, zirconium and mixtures of thereof. The binder may comprise a phosphate in the binder or a phosphate of the forenamed oxide binder materials. Preferably, the binder is a silicon oxide. The MFI zeolite may be supported in a silicon oxide containing binder or an alumina containing binder such as aluminum phosphate.

MFI zeolite slurry may be first mixed with a binder in the form of colloidal suspension (sol) and gelling reagent and then dropped into hot oil to make spheres controlled to produce 1/888-inch to about 1/32-inch diameter calcined supports. Alternatively, the zeolite may be mixed with a silicon oxide containing binder and extruded to 1/32 to ¼ inch diameter extrudates. Extrudates may be washed with ammonia to remove sodium ions from the zeolite, dried and calcined to remove the organic structural directing agent (OSDA) from the synthesized zeolite. Optionally, the calcined support may be ammonium-ion exchanged using an ammonium nitrate solution to remove residual sodium ions and dried at about 110° C.

The NEP catalyst comprises a metal on the catalyst. The metal may comprise a transition metal. In a further example, the metal may comprise platinum, palladium, iridium, rhenium, ruthenium and mixtures thereof. The metal may be a noble metal. A modifier metal may also be included on the catalyst. The modifier metal may include tin, germanium, gallium, indium, thallium, zinc, silver and mixtures thereof. The modifier metal should be more concentrated on the binder than on the zeolite. About 0.01 to about 5 wt % of each of the transition metal and the modifier metal may be on the catalyst.

Metal may be incorporated into the binder by evaporative impregnation. A solution of platinum such as tetraamine platinate nitrate or chloroplatinic acid may be contacted with the bound spherical or extrudate supports which have been calcined and ion-exchanged in a rotary evaporator, followed by drying and oxidation.

The NEP catalyst comprises a metal on the bound spherical or extrudate supports of the catalyst. Preferably, more of the metal is on the binder than on the zeolite. At least 60 wt %, suitably at least 70 wt %, preferably at least 80 wt % and most preferably at least 90 wt % of the metal is on the binder. The zeolite and/or the entire NEP catalyst is steamed oxidized to drive the metal off the zeolite. Steaming is preferably effected after the metal is added to the catalyst. The dried, impregnated spherical or extrudate supports may be steam oxidized in air for sufficient time to provide NEP catalysts. Steam oxidation in air at a temperature of about 500° C. to about 650° C. and about 5 mol % to about 30 mol % steam for about 1 to 3 hours may be suitable.

The NEP catalysts must be reduced to activate them for catalyzing the NEP reaction. For example, the catalyst may be reduced in flowing hydrogen at about 500 to about 550° C. for 3 hours before contacting feed.

After paraffin conversion, a light paraffinic stream is discharged from the NEP reactor 120 in an effluent line 122. The light paraffinic stream may comprise at least about 40 wt % ethane or at least about 40 wt % propane or at least about 70 wt % and preferably at least about 80 wt % ethane and propane. The ethane to propane ratio can range from about 0.1 to about 5. The light paraffinic stream can have less than about 16 wt %, suitably less than about 14 wt %, preferably less than about 12 wt %, and more preferably less than about 10 wt % methane.

The light paraffinic stream may be cooled and fed to an NEP separation unit 130. The NEP separation unit 130 may be a fractionation column or a series of fractionation columns and other separation units that may separate the light paraffinic stream in line 122 into the hydrogen stream in line 131, an ethane stream having a predominance of ethane in line 132, a propane stream having a predominance of propane in line 133 and a heavy aromatics stream in line 134. The NEP separation unit 130 may comprise a demethanizer column that separates the light paraffinic stream into a gas stream in an overhead line and a C2+ paraffin stream in a bottoms line. The gas stream may be sent to a hydrogen purification unit such as a PSA unit to recover hydrogen in line 131 for recycle to the NEP reactor 120 in line 166. Remaining methane from the hydrogen purification unit may be used for fuel gas. The C2+ paraffin stream may then be fed to a deethanizer column to produce the ethane stream in a deethanizer overhead line 132 and a C3+ paraffin stream in a deethanized bottoms line. The C3+ paraffin stream may then be fed to a depropanizer column to produce the propane stream in a depropanizer overhead line 133 and the heavy paraffin stream which may comprise C4+ hydrocarbons. The NEP separation unit 130 may take other forms.

For example, the NEP separation unit 130 may omit a demethanizer column and the light paraffinic stream in line 122 may feed a deethanizer column which produces a C2-stream in a deethanizer overhead line. The C2-stream can be separated in the hydrogen purification unit to recover a hydrogen stream in line 131 while residual ethane and methane from the hydrogen purification unit can comprise or supplement the ethane stream in line 132. The hydrogen purification unit may comprise a membrane unit and the hydrogen recovered from the membrane unit may be further purified in an absorption column before it is recycled to the NEP reactor 120 in lines 131 and 166. In an additional alternative, the C2-stream from the deethanizer column may be charged to an ethylene producing unit 140 in which ethane is converted to ethylene but methane and hydrogen rides through inertly to be recovered in a downstream ethylene recovery unit.

The ethane stream in line 132 may be charged to an ethylene producing unit 140 in which ethane in the ethane stream is converted into ethylene. In an embodiment, the ethylene producing unit 140 is a steam cracking unit. The ethane stream in line 132 may be cracked under steam in a furnace to produce a cracked stream including an ethylene stream 142. The ethane stream may be charged to the ethane steam cracking unit in the gas phase. The ethane steam cracking unit may preferably be operated at a temperature of about 750° C. (1382° F.) to about 950° C. (1742° F.). The cracked stream exiting the furnace of the ethane steam cracking unit may be in a superheated state. One or more quench columns, or other devices known in the art, but preferably an oil quench column and/or a water quench column, may be used for quenching or separating the cracked stream into a plurality of cracked streams. The ethane steam cracking unit may further comprise additional distillation columns, amine wash columns, compressors, expanders, etc. to separate the cracked stream into cracked streams rich in individual light olefins the most predominant of which is the ethylene stream in line 142. The ethylene stream may comprise a yield of at least 75 wt %, preferably at least 80 wt %, ethylene based on the ethane stream in line 132. Among the other components in the cracked stream exiting the ethane steam cracking, ethylene producing unit 140 may be hydrogen, methane, propylene, butene, and pyrolysis gas. Each of these components may be recovered and further processed.

The ethylene stream and a propylene stream from the ethylene producing unit 140 may be recovered or transported to polymerization plants, chemical plants or exported. A butene stream may be recovered and used to produce plastics or other petrochemicals by processes such as polymerization or exported. Product recovery of at least 50 wt %, typically at least 60 wt % and suitably at least 70 wt % of valuable ethylene, propylene, and butylene products is achievable from the ethane steam cracking unit 140 based on the ethane stream in line 132.

The propane stream in line 133 may be charged to a propylene producing unit 150 in which propane in the propane stream is converted into propylene. The propylene producing unit 150 may be a propane dehydrogenation (PDH) unit. PDH catalyst is used in a dehydrogenation reaction process to catalyze the dehydrogenation of propane. The conditions in the dehydrogenation reactor may include a temperature of about 500 to about 800° C., a pressure of about 40 to about 310 kPa (abs) and a catalyst to oil ratio of about 5 to about 100.

The dehydrogenation reaction may be conducted in a fluidized manner such that gas, which may comprise the reactant paraffins with or without a fluidizing inert gas, is distributed to the reactor in a way that lifts the dehydrogenation catalyst in the reactor vessel while catalyzing the dehydrogenation of paraffins. During the catalytic dehydrogenation reaction, coke is deposited on the dehydrogenation catalyst leading to reduction of the activity of the catalyst. The dehydrogenation catalyst must then be regenerated in a regenerator. The regenerator may combust coke from the dehydrogenation catalyst and fuel gas to ensure sufficient enthalpy in the dehydrogenation reactor to promote the endothermic reaction.

The dehydrogenation catalyst selected should minimize cracking reactions and favor dehydrogenation reactions. Suitable catalysts for use herein include an active metal which may be dispersed in a porous inorganic carrier material such as silica, alumina, silica alumina, zirconia, or clay. An exemplary embodiment of a catalyst includes alumina or silica-alumina containing gallium, a noble metal, and an alkali or alkaline earth metal.

The catalyst support comprises a carrier material, a binder and an optional filler material to provide physical strength and integrity. The carrier material may include alumina or silica-alumina. Silica sol or alumina sol may be used as the binder. The alumina or silica-alumina generally contains alumina of gamma, theta and/or delta phases. The catalyst support particles may have a nominal diameter of about 400 to about 5000 micrometers with the average diameter of about 600 to about 3500 micrometers. Preferably, the surface area of the catalyst support is about 85 to about 140 m²/g.

The fluidized dehydrogenation catalyst may comprise a dehydrogenation metal on a support. The dehydrogenation metal may be a one or a combination of transition metals. A noble metal may be a preferred dehydrogenation metal such as platinum or palladium. Gallium is an effective metal for paraffin dehydrogenation. Metals may be deposited on the catalyst support by impregnation or other suitable methods or included in the carrier material or binder during catalyst preparation.

The acid function of the catalyst should be minimized to prevent cracking and favor dehydrogenation. Alkali metals and alkaline earth metals may also be included in the catalyst to attenuate the acidity of the catalyst. Rare earth metals may be included in the catalyst to control the activity of the catalyst. Concentrations of 0.001% to 10 wt % metals may be incorporated into the dehydrogenation catalyst. In the case of the noble metals, it is preferred to use about 10 parts per million (ppm) by weight to about 600 ppm by weight noble metal. More preferably it is preferred to use about 10 to about 100 ppm by weight noble metal. The preferred noble metal is platinum. Gallium should be present in the range of 0.3 wt % to about 3 wt %, preferably about 0.5 wt % to about 2 wt %. Alkali and alkaline earth metals may be present in the range of about 0.05 wt % to about 1 wt %.

Regenerated catalyst may be contacted with the propane stream in line 26 perhaps with a fluidizing gas to lift the propane stream and dehydrogenation catalyst up a riser while dehydrogenation occurs. Above the riser spent dehydrogenation catalyst and propylene product may be separated by a centripetal separation device. Propylene product gas may be quenched with a cooling fluid to prevent over reaction to undesired by-products. Separation of the propylene product from the PDH effluent stream in line 152 may include quench contacting and fractionation to produce a propylene product stream. Unreacted propane may be recycled to the dehydrogenation reactor and lighter gases may be recycled to the regenerator as fuel gas to be combusted to provide enthalpy for the reaction.

The propylene producing unit may also employ a catalytic moving bed reactor. The reactor section may comprise several radial flow reactors in parallel or series heated by charge and interstage heaters. The propane stream perhaps with added hydrogen flows in each dehydrogenation reactor from a screened center pipe through an annular dehydrogenation catalyst bed to an outer effluent annulus. Flow may be in the reverse fashion. The dehydrogenation catalyst may comprise a noble metal or mixtures thereof, a modifier selected from the group consisting of alkali metals or alkaline-earth metals and mixtures thereof, a component selected from the group consisting of tin, germanium, lead, indium, gallium, thallium, and mixtures thereof, and a porous support forming a catalyst particle. The catalyst support may comprise oil dropped alumina spheres.

Dehydrogenation conditions may include a temperature of from about 400 to about 900° C., a pressure of from about 0.01 to 10 atmospheres absolute, and a liquid hourly space velocity (LHSV) of from about 0.1 to 100 hr⁻¹. The pressure in the dehydrogenation reactor is maintained as low as practicable, consistent with equipment limitations, to maximize chemical equilibrium advantages. Spent dehydrogenation catalyst in the annular catalyst bed may be withdrawn from the bottom of the bed, forwarded to a regenerator to combust coke from the catalyst with air at about 450 to about 600° C. Noble metal on the catalyst may be redispersed by an oxyhalogenation process, dried and returned to the top of the dehydrogenation catalyst bed as regenerated dehydrogenation catalyst.

Dehydrogenation effluent from the propylene producing unit 40 may be cooled, compressed, dried and hydrogen is cryogenically separated from the hydrocarbons with a net gas purity of 85 to 93 mol % hydrogen. Hydrocarbon liquid is selectively hydrogenated to convert diolefins and acetylenes and the hydrocarbon liquid is fractionated in a deethanizer column to remove ethane and propylene is split from propane in a propane-propylene splitter column to provide polymer-grade propylene. Propane may be recycled as feed to the propylene producing unit 40.

The heavy stream which may be taken from a bottom of a depropanizer column in line 134 may comprise C4+ paraffins. In an aspect, the heavy stream in line 134 may comprise greater than 98% aromatics.

In an embodiment, unconverted paraffins comprising C4 may be separated in the NEP separation unit 130 in line 137. The paraffins stream comprising C4 in line 137 may be recycled to the NEP reactor 120. The paraffins stream comprising C4 in line 137 may be combined with the charge stream in line 102 and passed to the NEP reactor in line 104.

The cracked stream from the ethylene producing unit 140 may be passed to the product recovery unit 160 for separating ethylene from other components. The PDH effluent stream in line 152 may also be passed to the product recovery unit 160 for separating propylene from other components. The product recovery unit 160 may comprise quenching and fractionation to separate the ethylene and propylene. An ethylene stream having a predominance of ethylene is separated in line 164 from the product recovery unit 160 and a propylene stream having a predominance of propylene is taken in line 165 from the product recovery unit 160. Hydrogen is separated and taken in line 161 from the product recovery unit 160. The hydrogen stream in line 161 may be recycled to the NEP reactor 120. In an embodiment, the hydrogen stream in line 161 may be separated to provide a hydrogen recycle stream in line 163. The remaining hydrogen can be taken in line 162 for further use or separated. The hydrogen recycle stream in line 163 may be combined with the hydrogen stream in line 131 to provide a combined hydrogen recycle stream in line 166 which is passed to the NEP reactor 120. In an aspect, the combined hydrogen recycle stream in line 166 may be combined with the charge stream and passed to the NEP reactor 120 in line 104.

Usually, ethane from the NEP process is fed to an ethane steam cracker and propane is fed to PDH unit. The run length for ethane steam crackers between major turn-arounds may typically include more than five years. The run length for the PDH unit between major turn-arounds is less than five years, such as three years. The turn-around times for the PDH unit may last for several weeks. During such events when the PDH unit is down, it is highly desirable for the ethane steam cracker not to be shut down since it would negatively impact operating profits. In other words, the NEP unit needs to keep running to supply ethane to the ethane steam cracking unit. However, the NEP unit continues to produce propane. When the PDH unit is down, the propane needs an alternative use. Storing the produced propane may need more than 30 MMTA of storage. This much storage would be very expensive. Moreover, there may be times when propylene demand is sufficiently low relative to ethylene demand to incentivize a production of much greater ethane than propane.

The present process includes recycling the propane back to the NEP reactor when the PDH is down because the propylene producing unit 150 is no longer processing the propane or it is processing much less propane. The process provides a cost-effective solution for times when the propylene producing unit 150 is down scenario that keeps the ethane steam cracker running or enable higher ethane: propane (E:P) ratios. The disclosed process with propane recycle is expected to lower capital expenditure and provide a most economical solution for times when the PDH is down.

In accordance with the present disclosure, the propane stream in line 133 may be separated to provide a propane recycle stream in line 136 and a propane feed stream in line 135. The propane feed stream in line 135 may be passed to the propylene producing unit 150 or PDH 150. The propane recycle stream in line 136 may be combined with the naphtha stream in line 102 and charged to the NEP reactor 120 in charge line 104. When the propane recycle stream in line 136 is recycled to the NEP reactor, the flow rate of the naphtha stream in line 102 to the NEP reactor 120 may be regulated via the control valve 10 to account for the flow rate of the propane recycle stream in line 136. In an exemplary embodiment, the flow rate of the naphtha stream in line 102 is decreased via the control valve 10 proportional to the increase in the flow rate of the propane recycle stream to the NEP reactor 120. In times when the propylene producing unit 150 is down, the flow of the propane feed stream in line 135 to the propylene producing unit 150 is stopped by completely closing the control valve 20 on the propane feed line 135. In times when the propylene producing unit 150 is down, the propane stream in line 133 is recycled in its entirety to the NEP reactor 120 via the propane recycle stream in line 136. In times when the propylene producing unit 150 is down, the flow of the naphtha stream in line 102 may be decreased to a proportionally lower flow rate by partially closing the control valve 10. In times when the propylene producing unit 150 is running to produce the propylene, the propane stream in line 133 may be split to provide both the propane feed stream in line 135 and the propane recycle stream in line 136. Recycling the propane back to the NEP reactor 120 when the propylene producing unit 150 is running would provide additional propane to the NEP reactor 120 to make more ethane which is fed to the ethane conversion unit 140.

In accordance with the present disclosure, recycling the propane recycle stream in line 136 to the NEP reactor 120 may include adjusting the NEP reactor inlet temperatures to optimize the overall conversion and the process 101. In accordance with the present disclosure, the propane recycle stream in line 136 may be recycled to the NEP reactor at an inlet temperature of about 425° C. to about 525° C.

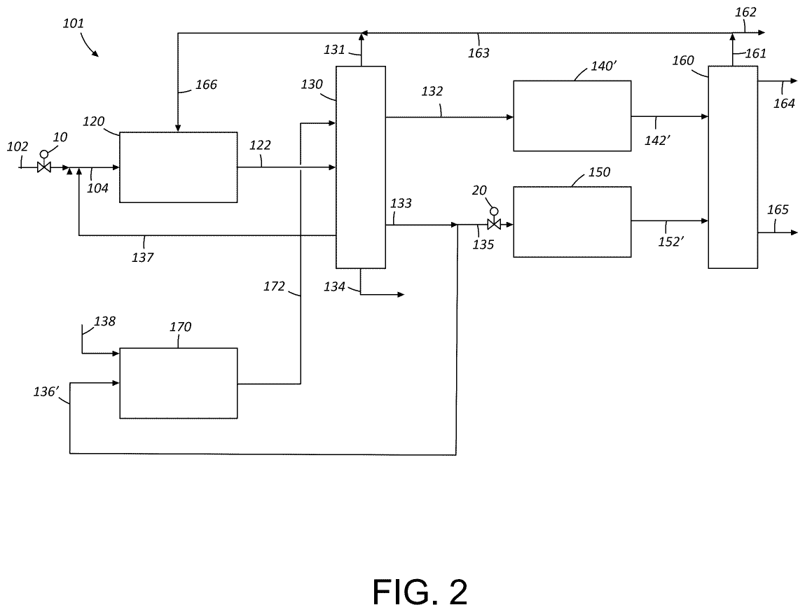

FIG. 2 shows an alternative embodiment to the embodiment of FIG. 1 which employs a dedicated propane to ethane reactor 170. Elements in FIG. 2 with the same configuration as in FIG. 1 will have the same reference numeral as in FIG. 1. Elements in FIG. 2 which have a different configuration as the corresponding element in FIG. 1 will have the same reference numeral but designated with a prime symbol (′). The configuration and operation of the embodiment of FIG. 2 is essentially the same as in FIG. 1 with the following exceptions.

The propane recycle stream in line 136′ can be converted to ethane in a dedicated propane to ethane (PE) reactor. The propane recycle stream in line 136′ is passed to a dedicated propane to ethane reactor 170. A hydrogen stream in line 138 may be passed to the propane to ethane reactor 170. In an aspect, the hydrogen stream in line 138 may be taken from the hydrogen stream in line 166. In the propane to ethane reactor 170, the propane recycle stream in line 136′ and the hydrogen stream in line 138 are contacted with a PE catalyst to convert the propane to ethane. In an exemplary embodiment, the propane to ethane reactor 170 may comprise the NEP catalyst used in the NEP reactor 120. In another embodiment, the NEP catalyst used in the PE reactor 170 may be slightly different so attuned to convert propane to ethane. An ethane stream in line 172 may be taken from the propane to ethane reactor 170. The ethane stream in line 172 may be passed to the NEP separation unit 130 and separated along with the light paraffinic stream in line 122 from the NEP reactor 120. The benefit of a dedicated propane to ethane reactor includes lower coke formation.

In the times when the propylene producing unit 150 is down, the flow of the propane feed stream in line 135 to the PDH unit 150 is stopped by completely closing the control valve 20 on the propane feed line 135. In times when the propylene producing unit 150 is down, the propane stream in line 133 is recycled in its entirety to the propane to ethane reactor 170 via the propane recycle stream in line 136′. In times when the propylene producing unit 150 is down, passing the propane recycle stream in line 136′ to the dedicated propane to ethane reactor 170 may not affect the throughput of the naphtha stream in line 102. The rest of the process is the same as previously described.

### Example

A comparative study was performed to compare the process with propane recycle with a process producing ethane and propane without propane recycle. The feed rate and the results of the two processes are as below in Table:

TABLE

| | Without propane recycle | Propane recycle |
|---|---|---|
| Naphtha Feed (kMTA) | 670 | 515 |
| Hydrogen (kMTA) | 30 | 27 |
| Ethane (kMTA) | 400 | 400 |
| Propane (kMTA) | 150 | NA |

As evident form the results in the table, 670 kMTA of the naphtha feed was required to produce 400 kMTA of ethane when propane was not recycled. With propane recycle, only 515 kMTA of the naphtha feed was required to produce the same amount of 400 kMTA of ethane. Thus, the process with propane recycle consumed much lower amount of feed, more than 23% less feed, than the process with no propane recycle for the same amount of the product ethane. So, it is preferable to recycle the propane back to the reactor when a propane product stream is not required.

### SPECIFIC EMBODIMENTS

While the following is described in conjunction with specific embodiments, it will be understood that this description is intended to illustrate and not limit the scope of the preceding description and the appended claims.

A first embodiment of the present disclosure is a process for converting naphtha comprising contacting a naphtha stream and a hydrogen stream with a catalyst in a reactor to produce a light paraffinic stream; separating the light paraffinic stream into an ethane stream and a propane stream; and recycling a propane recycle stream taken from the propane stream to the reactor. An embodiment of the present disclosure is one, any or all of prior embodiments in this paragraph up through the first embodiment in this paragraph further comprising taking a propane feed stream from the propane stream; and charging the propane feed stream to a dehydrogenation unit to produce propylene. An embodiment of the present disclosure is one, any or all of prior embodiments in this paragraph up through the first embodiment in this paragraph wherein entirety of the propane stream is recycled to the reactor. An embodiment of the present disclosure is one, any or all of prior embodiments in this paragraph up through the first embodiment in this paragraph further comprising decreasing a flow rate of the naphtha stream to the reactor based on a flow rate of the recycled propane stream to the reactor. An embodiment of the present disclosure is one, any or all of prior embodiments in this paragraph up through the first embodiment in this paragraph further comprising separating a C4 stream from the light paraffinic stream; and passing the C4 stream to the reactor. An embodiment of the present disclosure is one, any or all of prior embodiments in this paragraph up through the first embodiment in this paragraph further comprising terminating the charging of the propane feed stream to the dehydrogenation unit. An embodiment of the present disclosure is one, any or all of prior embodiments in this paragraph up through the first embodiment in this paragraph further comprising converting ethane in the ethane stream into ethylene. An embodiment of the present disclosure is one, any or all of prior embodiments in this paragraph up through the first embodiment in this paragraph further comprising charging the ethane stream to a steam cracking unit. An embodiment of the present disclosure is one, any or all of prior embodiments in this paragraph up through the first embodiment in this paragraph further comprising charging the propane recycle stream to a dedicated propane to ethane reactor. An embodiment of the present disclosure is one, any or all of prior embodiments in this paragraph up through the first embodiment in this paragraph further comprising separating the light paraffinic stream into a methane hydrogen stream and recycling the hydrogen stream back to the contacting step. An embodiment of the present disclosure is one, any or all of prior embodiments in this paragraph up through the first embodiment in this paragraph wherein the propane recycle stream is recycled to the reactor at an inlet temperature of about ° C. to about ° C.

A second embodiment of the present disclosure is a process for converting naphtha comprising contacting a naphtha stream and a hydrogen stream with a catalyst in a reactor to produce a light paraffinic stream; separating the light paraffinic stream into an ethane stream and a propane stream; recycling a propane recycle stream taken from the propane stream to the reactor; and decreasing a flow rate of the naphtha stream to the reactor based on a flow rate of the recycled propane stream to the reactor. An embodiment of the present disclosure is one, any or all of prior embodiments in this paragraph up through the second embodiment in this paragraph further comprising taking a propane feed stream from the propane stream; and charging the propane feed stream to a dehydrogenation unit to produce propylene. An embodiment of the present disclosure is one, any or all of prior embodiments in this paragraph up through the second embodiment in this paragraph wherein entirety of the propane stream is recycled to the reactor. An embodiment of the present disclosure is one, any or all of prior embodiments in this paragraph up through the second embodiment in this paragraph further comprising terminating the charging of the propane feed stream to the dehydrogenation unit. An embodiment of the present disclosure is one, any or all of prior embodiments in this paragraph up through the second embodiment in this paragraph further comprising converting ethane in the ethane stream into ethylene. An embodiment of the present disclosure is one, any or all of prior embodiments in this paragraph up through the second embodiment in this paragraph further comprising charging the ethane stream to a steam cracking unit.

A third embodiment of the present disclosure is a process for converting naphtha comprising contacting a naphtha stream and a hydrogen stream with a catalyst in a reactor to produce a light paraffinic stream; separating the light paraffinic stream into an ethane stream and a propane stream; and recycling the entire propane stream to the reactor to produce a net ethane stream from the reactor. An embodiment of the present disclosure is one, any or all of prior embodiments in this paragraph up through the third embodiment in this paragraph further comprising terminating charging of a propane feed stream taken from the propane stream to a dehydrogenation unit. An embodiment of the present disclosure is one, any or all of prior embodiments in this paragraph up through the third embodiment in this paragraph further comprising decreasing a flow rate of the naphtha stream to the reactor based on a flow rate of the propane stream to the reactor.

Without further elaboration, it is believed that using the preceding description that one skilled in the art can utilize the present disclosure to its fullest extent and easily ascertain the essential characteristics of this disclosure, without departing from the spirit and scope thereof, to make various changes and modifications of the disclosure and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

1. A process for converting naphtha comprising:
contacting a naphtha stream and a hydrogen stream with a catalyst in a reactor to produce a light paraffinic stream;
separating the light paraffinic stream into an ethane stream and a propane stream; and
recycling a propane recycle stream taken from the propane stream to the reactor.

2. The process of claim 1 further comprising:
taking a propane feed stream from the propane stream; and
charging the propane feed stream to a dehydrogenation unit to produce propylene.

3. The process of claim 1 wherein entirety of the propane stream is recycled to the reactor.

4. The process of claim 1 further comprising decreasing a flow rate of the naphtha stream to the reactor based on a flow rate of the recycled propane stream to the reactor.

5. The process of claim 1 further comprising:
separating a C4 stream from the light paraffinic stream; and
passing the C4 stream to the reactor.

6. The process of claim 3 further comprising terminating the charging of the propane feed stream to the dehydrogenation unit.

7. The process of claim 1 further comprising converting ethane in said ethane stream into ethylene.

8. The process of claim 7 further comprising charging said ethane stream to a steam cracking unit.

9. The process of claim 1 further comprising charging said propane recycle stream to a dedicated propane to ethane reactor.

10. The process of claim 1 further comprising separating said light paraffinic stream into a methane hydrogen stream and recycling the hydrogen stream back to the contacting step.

11. The process of claim 1 wherein the propane recycle stream is recycled to the reactor at an inlet temperature of about 425° C. to about 525° C.

12. A process for converting naphtha comprising:
contacting a naphtha stream and a hydrogen stream with a catalyst in a reactor to produce a light paraffinic stream;
separating the light paraffinic stream into an ethane stream and a propane stream;
recycling a propane recycle stream taken from the propane stream to the reactor; and
decreasing a flow rate of the naphtha stream to the reactor based on a flow rate of the recycled propane stream to the reactor.

13. The process of claim 12 further comprising:
taking a propane feed stream from the propane stream; and
charging the propane feed stream to a dehydrogenation unit to produce propylene.

14. The process of claim 12 wherein entirety of the propane stream is recycled to the reactor.

15. The process of claim 14 further comprising terminating the charging of the propane feed stream to the dehydrogenation unit.

16. The process of claim 12 further comprising converting ethane in said ethane stream into ethylene.

17. The process of claim 16 further comprising charging said ethane stream to a steam cracking unit.

18. A process for converting naphtha comprising:
contacting a naphtha stream and a hydrogen stream with a catalyst in a reactor to produce a light paraffinic stream;
separating the light paraffinic stream into an ethane stream and a propane stream; and
recycling the entire propane stream to the reactor to produce a net ethane stream from the reactor.

19. The process of claim 18 further comprising:
separating a C4 stream from the light paraffinic stream; and
passing the C4 stream to the reactor.

20. The process of claim 18 further comprising decreasing a flow rate of the naphtha stream to the reactor based on a flow rate of the propane stream to the reactor.
